(19) United States
(12) Patent Application Publication
(10) Pub. No.: US 2002/0137210 A1
(43) Pub. Date: Sep. 26, 2002

(54) Method for modifying genetic characteristics of an organism

(76) Inventor: Nikolai Andreevich Churikov, Moscow (RU)

Correspondence Address: BURNS DOANE SWECKER & MATHIS L L P, POST OFFICE BOX 1404, ALEXANDRIA, VA, 22313-1404, US

(21) Appl. No.: 09/913,040

(22) PCT Filed: Dec. 7, 2000
(86) PCT No.: PCT/RU00/00504

(30) Foreign Application Priority Data
Dec. 9, 1999 (RU) 99125827

(51) Int. Cl. A61K 48/00; C12N 15/87

(52) U.S. Cl. 435/455; 514/44

(57) ABSTRACT

The invention concerns with the molecular biology, molecular genetics and biotechnology and can be used in the gene-therapy in the medicine and the agriculture or in the industrial biotechnology for a gene-specific silencing of the disease-related genes or the genes interfering a buildup of a product, respectively.

The approach is suggested for changing of genetic properties of an organism by RNA interference leading to gene-specific silencing of a selected gene by RNA molecules, that are complementary in a parallel orientation (pcRNA) to mRNA of the selected gene; pcRNA are synthesized in vivo or in vitro on the artificial DNA sequence possessing symmetrical nucleotide ordering (mirror inversion) in respect to the nucleotide sequence of the gene.

The invention suggests the general approach for changing of genetic properties of an organism and is based on the biological properties of mirror inversions of nucleotide sequences that are realized in RNA interference and gene-specific silencing.

### FIELD OF INVENTION

The invention concerns with the molecular biology, molecular genetics and biotechnology and can be used in the gene-therapy in the medicine and the agriculture or in the industrial biotechnology for a gene-specific silencing of the disease-related genes or the genes interfering a buildup of a product, respectively.

### BACKGROUND OF INVENTION

Different approaches for changing the genetic properties of an organism are used. Some of them assume the damage of a gene. The example of the latter is so-called genes' knockout. The approach utilizes the damage (mutation) of a selected gene in germ-line or stem cells and thus cannot be used in the most cases for the developed organism [L. V. Varga, S. Toth, I. Novak, A. Falus, Immunol. Lett., 1999, vol. 69, p. 217; J. Osada, N. Maeda, Methods Mol. Biol., 1998, vol. 110, p. 79].

During recent years an increased attention is attracted by another approach for changing the genetic properties of an organism by RNA interference, leading to a gene-specific silencing by changes of the regulation of an undamaged gene [M. K. Montgomery, A. Fire, Trends in Genetics, 1998, vol.14, p. 255; P. Sharp, Genes & Development, 1999, vol.13, p. 139]. RNA interference can be used for gene-specific silencing at any stage of development, including the adults. The increased attention to RNA interference is due to the fact that these studies serendipitously uncovered the ancient mechanisms of gene regulation. The physiological role of this mechanism of regulation could include the local changes of chromosomal structures, transcription activity, RNA processing and transport into the cytoplasm and RNA stability.

Up-to-now RNA interference leading to gene-specific silencing was described in different organisms—nematode, Drosophila, fungi, plants.

The known approach for changes of genetic properties of an organism, that is based on RNA interference, uses the antisense RNA (asRNA) that is complementary to the mRNA of the selected gene in antiparallel orientation and is synthesized in vitro and introduced into organism [A. Fire, S-Q. Xu, M. K. Montgomery, S. V. Kostas, S. E. Driver, C. C. Mello, Nature, 1998, vol. 391, p. 806].

The described approach is carried out as follows:

1. A gene with pathogenic activity is selected;

2. A DNA construct possessing a selected gene or its cDNA (a sequence corresponding to mRNA), i.e. natural DNA, in the opposite polarity under the control of selected promoter is prepared. This permits to perform transcription of non-coding strand of the gene. For the generation of the construct different vectors are used possessing DNA sequences for selection of transformants, for efficient expression of the turned-over gene and for correct “inscribing” of the construct into chromosomal domains;

3. asRNA is synthesized in vitro on the construct and introduced into organism by different methods (electroporation, injections, per os).

An important problem of this approach for changing of genetic properties of an organism by RNA interference is the often occurrence of reversions of the constructs designed for asRNA synthesis by rearrangements leading to stop of asRNA transcription and start of transcription of the sequences corresponding to mRNA-strand. Thus, instead of inhibition of activity of the selected gene an increased transcription of the gene could occur. Start of transcription of the sequences corresponding to mRNA-strand can also happen if in the target site of insertion of the construct the host promoter sequences transcribing the sense strand are present. The probability of such events is rather high.

The highness of reversions is illustrated by demonstrative experiments on transgenic organisms. The constructs in these cases were introduced with the opposite aim—to increase the activity of a selected gene. However, reversions by spontaneous activation of transcription from the opposite strand resulted in complete inhibition of gene activity instead of activation of its expression, i.e. to gene-specific silencing by RNA interference mechanisms [M. K. Montgomery, A. Fire, Trends in Genetics, 1998, vol.14, p. 255; P. Sharp, Genes & Development, 1999, vol.13, p.139].

### DISCLOSURE OF THE INVENTION

The approach is suggested for changing of genetic properties of an organism by RNA interference leading to gene-specific silencing of a selected gene by RNA molecules, that are complementary in a parallel orientation (pcRNA) to mRNA of the selected gene; pcRNA are synthesized in vivo or in vitro on the artificial DNA sequence possessing symmetrical nucleotide ordering (mirror inversion) in respect to the nucleotide sequence of the gene.

### BRIEF DESCRIPTION OF THE DRAWINGS

FIG. 1 shows the structure of the DNA construct possessing mirror nucleotide sequence in respect the E. coli lon gene and designed for expression of pcRNA, where:

a—relationship between nucleotide sequence of the Ion gene and the artificial chemically synthesized DNA possessing mirror nucleotide sequence in respect the gene (+ strands correspond to parlon pcRNA or mRNA); the synthesized DNA in the construct is under the control of the lac promoter driving the expression of parlon pcRNA;

b—relationship between lon mRNA and parlon pcRNA;

c—genes in the lux-regulon: pr and pl promoters of the lux-regulon are in different strands, genes are shown by the open bars; Lon protease cleaves the gene R product, that activates pr, and thus switches out the genes involved in the luminescence.

FIG. 2 shows the luminescence of E. coli cells that was observed after silencing of the ion gene induced by expression of parlon pcRNA, where:

lux-regulon from Vibrio fischeri was introduced into lon⁺ or lon⁻⁰ E. coli cells. In the resulted lon⁺ cells either the construct prepared in the pUC12 vector expressing parlon pcRNA or original pUC12 vectors without any insertion were introduced. The dependence of luminescence of the cells suspension presented as luminometer data in μV from optical density (OD₅₅₀). Silencing of the lon gene induces the luminescence of the transformants expressing parlon construct.

FIG. 3 shows the structure of the DNA conctruct possessing mirror nucleotide sequence in respect the Drosophila Kruppel gene and designed for expression of pcRNA (Kr-par), where:

a—relationship between nucleotide sequence of the Kruppel gene and the artificial chemically synthesized DNA, possessing mirror nucleotide sequence in respect the gene (+ strands correspond to Kr-par pcRNA or mRNA); the synthesized DNA in the construct is under the control of the T7 RNA polymerase promoter driving the expression of Kr-par pcRNA (synthesis on the opposite strand of the construct is drived by SP6 RNA polymerase promoter);

b—relationship between Kruppel mRNA and Kr-par pcRNA.

FIG. 4. shows the phenotypes of normal larva and Kr phenocopies generated after injections of the Kr-par pcRNA, where:

a—phenotypes of normal Drosophila larva;

b—phenotype of the larva developed after injection of the Kr-par pcRNA and possessing deletions of adjacent thoracic and the first abdominal segments;

c—phenotype of the larva developed after injection of the Kr-par pcRNA and possessing deletions of adjacent thoracic and three anterior abdominal segments.

Arrow shows the atopic tracheal ending that is characteristic only for Kr phenotype.

### BEST MODE OF CARRYING OUT THE INVENTION

The basis of the suggested invention was to increase the RNA interference reliability and to exclude the possibility of reversions leading to the synthesis of mRNA sequences on a construct designed for gene-specific silencing.

The approach is suggested for changing of genetic properties of an organism by RNA interference leading to gene-specific silencing of a selected gene by RNA molecules that are complementary in a parallel orientation (pcRNA) to mRNA of the selected gene; pcRNA are synthesized in vivo or in vitro on the artificial DNA sequence possessing symmetrical nucleotide ordering (mirror inversion) in respect to the nucleotide sequence of the gene.

This approach leads to more efficient RNA interference and gene-specific silencing and excludes the synthesis of mRNA sequences because in the constructs the non-homologous artificial DNA sequence is used.

The suggested approach is carried out as follows:

1. A gene with pathogenic activity (leading to a disease or interfering a buildup of biotechnological product) is selected;

2. Artificial DNA sequence is chemically synthesized, possessing the mirror nucleotide ordering in respect to the selected gene or its fragment;

3. A DNA construct is prepared on the basis of different vectors, possessing the chemically synthesized DNA sequence under the control of appropriate promoter, and a number of sequences important for efficient expression of the insert, and for correct “inscribing” of the construct in chromosomal domains;

4. The construct is introduced into organism by different methods for in vivo synthesis of pcRNA (transformation), or pcRNA is synthesized in vitro and used for injections.

The invention is clarified by the examples describing the realization of the suggested approach for changing of genetic properties of organisms (in which the changes of phenotype are observed) and illustrated by 4 figures.

### THE DATA SUPPORTING THE FEASIBILITY OF THE INVENTION

The following examples are preferable and aimed only to confirm the feasibility of the invention and cannot be used as an argument for the restriction of the capacity of the Inventor's claims. Expert in the field will find easily another applications for the invention, which are undoubtedly covered by the Inventor's claims stated in the Claim listed below.

### EXAMPLE 1

### RNA Interference in Escherichia coli Induced by in vivo Synthesized RNA Molecules that are Complementary in the Same Polarity to mRNA of the lon Gene

The lon gene was selected as far as it plays a key role in different regulatory events in E. coli cells.

Artificial DNA 95-bp (bp—base pair in DNA or RNA) sequence possessing the mirror nucleotide ordering in respect to the region of the lon gene was chemically synthesized.

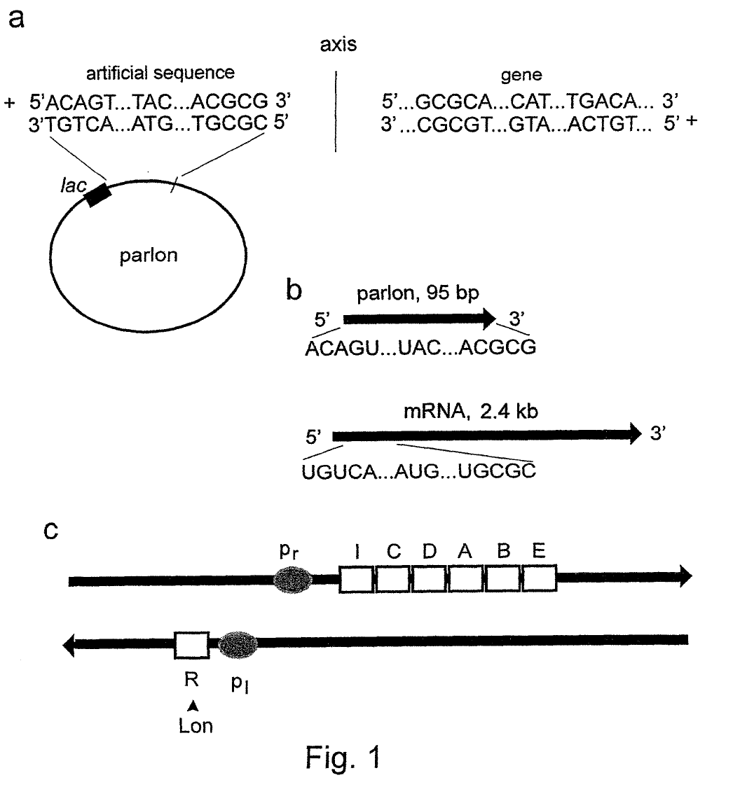

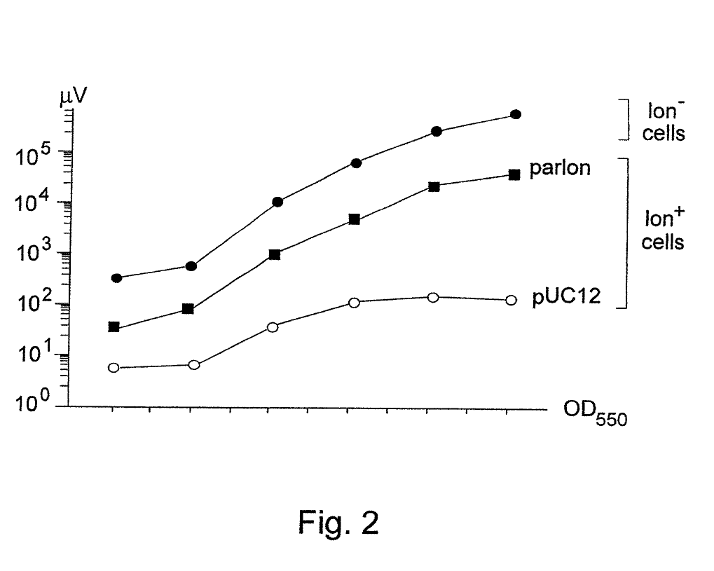

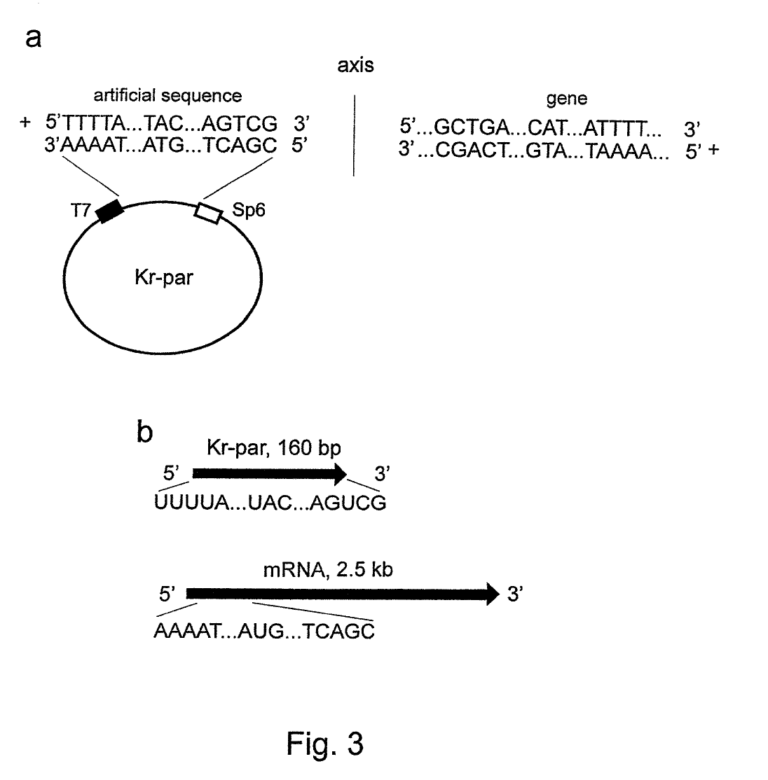

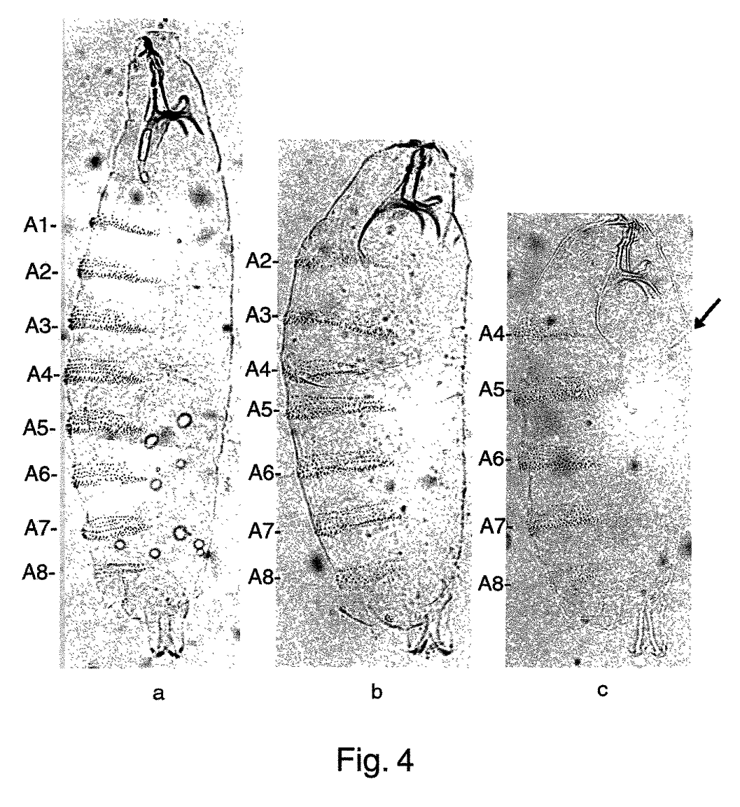

This DNA was used for the pUC12-construct in which the strand expressing the parlon pcRNA is under the control of the lac promoter (FIGS. 1a, b).

The construct was introduced into E. coli cells by transformation.

The impact of parlon pcRNA expression on the activity of the endogenous lon gene was measured by effect of the latter on the activity of the lux-regulon that was introduced into E. coli cells from Vibrio fischeri. Lon protease is a negative regulator of the lux-regulon because it specifically cleaves the LuxR protein. The latter forms the complex with autoinductor and finally activates expression of the proteins involved in the luminescence. Promoters pr and pl of the lux-regulon are located in different strands, genes involved in the luminescence are shown by the open bars (FIG. 1c). Actively expressing lon gene inhibits the transcription in the lux-regulon, that phenotypically is observed as inhibition of the luminescence. In contrast, silencing of the lon gene leads to increase of the LuxR concentration and to activation of the transcription in the lux-regulon and, consequently, to considerable enhancement of the luminescence of the cells. Lux-regulon, as 16 kb BamHI DNA fragment from Vibrio fischer, i was introduced into lon⁺ cells E. coli K12 AB1157 or lon⁻ cells E. coli K12 AB1899 (lonl).

The resulted lon⁺ cells are transformed either by the pUC12-construct expressing parlon pcRNA, or by pUC12 vector without any insert. Silencing of the lon gene is measured by enhancement of the luminescence of the cells. The luminescence of the cells expressing the parlon pcRNA is increased in several orders of the magnitude in comparison with the control pUC12-containing cells (FIG. 2).

The parlon pcRNA expressing transformants grown on plates reveal the characteristic property of the lon⁻ phenotype or for the silenced lon gene and form rather mucous colonies.

### EXAMPLE 2

### Generation of Kruppel (Kr) Phenocopies by Injections into Drosophila Embryos of in vitro Synthesized RNA, that is Complementary in the Same Polarity to mRNA

Kr is a homeotic gene, that is active in zygote and controls segment formation at the early embryonic stage of Drosophila development, was selected as a model allowing one to observe the early development in a multicellular organism. Kr mutants have deletions of the adjacent thoracic and anterior abdominal segments. Phenotypically this is observed just after cuticula formation and hatching of the larvae. Kr mutants have deletions of the adjacent thoracic and from one to several anterior abdominal segments and, sometimes, the development of actopic tracheal ending in the anterior part of the larva [E. Weischaus, C. Nusslein-Volhard, H. Kluding, Development, 1984, vol. 104, p. 172]. Thereafter, the Kr mutants have a unique phenotype developed as early as one day, that gives an advantage for study of the effect of RNA on a phenotype. Additional argument in favor of this model was that the study by antisense RNA injections was performed earlier [U. B. Rosenberg, A. Preiss, E. Seifert, H. Jackle, D. C. Knippe, Nature, 1985, vol. 313, p. 703].

Artificial DNA 160-bp sequence possessing the mirror nucleotide ordering in respect to the region of the Kr gene was chemically synthesized (FIGS. 3a, b). It should be stressed that antisense RNAs are synthesized on the non-coding strand of the same gene, while pcRNA can be synthesized only on the heterologous artificial DNA possessing the mirror order of nucleotide sequence.

The chemically synthesized artificial DNA is used for the construct in the pGEM-1 vector allowing to perform pcRNA synthesis with T7 RNA polymerase. pcRNA was denoted as Kr-par because it is complementary in a parallel orientation to Kr mRNA.

Embryos of the Oregon RC line are injected with pcRNA samples in the posterior pole at the syncytial stage and incubated under the water at 25° C. for 18-24 h. Then cuticula mounts are prepared and studied under the phase-contrast microscope.

The development of the larvae possessing typical Kr phenotype were observed. In control experiments, after injections of RNA synthesized on the opposite strand of the same construct with SP6 RNA polymerase, the development of normal larvae were observed.

FIG. 4 shows the normal larva (a) and two larvae developed after injections of Kr-par preparation (b, c). The latter have deletions of the thoracic and one or three abdominal segments and actopic tracheal ending in the anterior part of the larva, that is typical for Kr phenotype. The frequencies of observed phenocopies are about the same value as after injections with asRNA [U. B. Rosenberg, A. Preiss, E. Seifert, H. Jackle, D. C. Knippe, Nature, 1985, vol. 313, p. 703]. This demonstrats that pcRNA effects the expression of a key gene for differentiation in a multicellular organism, and a directed change of genetical properties of organism is attained.

Thus, the effect of nucleic acids possessing mirror inversions of nucleotide sequences and introduced by different ways in an organism on the phenotype is revealed. The main advantage of the suggested approach is that mirror sequences are capable of pcRNA synthesis and RNA interference but being heterologous cannot specify the corresponding mRNA sequence. That is why the utilization of this approach (“palindromic” approach), in contrast to regular approach using asRNA (“antisense” approach), cannot lead to reversion and to synthesis of the mRNA sequence.

### INDUSTRIAL APPLICABILITY

The invention suggested the general approach for changing of genetic properties of an organism and is based on the biological properties of mirror inversions of nucleotide sequences that are realized in RNA interference and gene-specific silencing. The suggested approach is based on the potent and specific biological activity of the transcripts coming from mirror inversions of nucleotide sequences in DNA, leads to changes in phenotype, and can be used in the gene-therapy in the medicine and the agriculture or in the industrial biotechnology for a gene-specific silencing of the disease-related genes or the genes interfering a buildup of a product, respectively.

1. The approach for changing of genetic properties of an organism by RNA interference leading to gene-specific silencing of a selected gene by RNA molecules, that are synthesized in vitro, differing by usage of RNA molecules that are complementary in a parallel orientation (pcRNA) to mRNA of the selected gene; pcRNA are synthesized in vivo or in vitro on the artificial DNA sequence possessing symmetrical nucleotide ordering (mirror inversion) in respect to the nucleotide sequence of the gene.
